(19) United States
(12) Patent Application Publication
(10) Pub. No.: US 2009/0217822 A1
(43) Pub. Date: Sep. 3, 2009

(54) Method and apparatus for controlling the amount of dissolved gas in a liquid

(76) Inventor: Joseph Kent Cyr, Milford, MA (US)

Correspondence Address: Joseph K. Cyr, 7 Ragged Hill Road, Milford, MA, 01757-1029, US

(21) Appl. No.: 12/074,126

(22) Filed: Feb. 29, 2008

(51) Int. Cl. C12M 1/107; B01F 3/04

(52) U.S. Cl. 99/276; 426/231; 435/289.1; 137/209; 137/213

(57) ABSTRACT

This is a method for regulating pressure inside of a vessel containing a fermenting liquid for the purpose of regulating the amount of dissolved gas in the liquid. This is achieved by replacing the safety relief valve spring on the pressure vessel with a selected spring of known tension to maintain the desired pressure in the vessel. The method will vent excess pressure in the vessel automatically by having the spring compress more under the increased tension from the relief valve, thereby opening the valve and releasing excess gas until the pressure drops to the desired level and closing the valve.

### FIELD OF THE INVENTION

Implementations consistent with the principles of the invention relate generally to a process which uses springs for pressure regulation and control of gas solubility in a liquid.

### BACKGROUND OF THE INVENTION

Pressure relief devices are used under a variety of applications, mostly for safety purposes to prevent explosion of pressurized vessels and devices. However, there are times when a more controlled pressure is desired and not for purposes of safety but in the finer regulation of pressure, for example, to control or maintain the volume of dissolved gas in a liquid.

The solubility of gasses in a liquid are generally a function of the temperature of the liquid, the type of gas involved and the pressure applied to the headspace (the area above the liquid) containing the gas in an enclosed vessel. By varying the parameters of the pressure relief device, the maximum volume of gas absorbed in the liquid at a given temperature can be controlled, up to a maximum that a set pressure will allow over time. This pressure-controlled process becomes important, especially when the applied pressure is being generated by an uncontrolled process, such as fermentation, from within the enclosed vessel. Excess gas will be discharged automatically when the pressure rises above the set point, the pressure will remain relatively constant and the dissolved gas volume will be predictable for a given temperature and type of gas.

Some other methods of regulating gas pressure inside this closed vessel where gas pressure is generated from within usually are either not done and the vessel is vented to the atmosphere or performed by manually venting the vessel, such as opening a bleeder valve, while monitoring the pressure with a pressure gauge. This manual process may not be very accurate or consistent. The frequency of venting off excess gas and the accuracy of the venting process are areas of concern if a predictable dissolved gas volume is desired. Not only is this manual process time consuming, it lends itself to quality control issues including over pressurization; therefore too much gas is absorbed. This may require another time consuming manual process to remove the excess gas. In general, the quality of the end product may be compromised or at the very least, may be inconsistent from one production run to another.

Another process where automated, controlled, gas pressure regulation is important is when a pressurized liquid containing dissolved gasses is transferred from one vessel to another. Manually controlling the pressure during the transfer process is time consuming and subject to error. For example, inadvertent losses of pressure during the transfer process will likely release dissolved gasses, compromising the quality of the end product by reducing the amount of dissolved gasses in the liquid. On the other hand, too much additional pressure during the transfer of liquid from one pressure vessel to another will likely add additional dissolved gasses to the end product. In either case, the end product has changed with respect to the amount of dissolved gasses in the liquid. The use of transfer pumps would likely cause a reduction in dissolved gas because there is generally a large differential in pressure from the inlet to the outlet of a pump. This pressure reduction on the pump inlet may force dissolved gasses from solution possibly causing a loss of prime and flow of liquid and may still require an automated pressure relief system on the receiving vessel to prevent a potentially dangerous over pressurization condition.

### SUMMARY OF THE INVENTION

In accordance with an implementation, a spring-controlled relief valve, which uses interchangeable springs of various tensions to set different relief pressures, or uses an adjustable spring to set relief pressure, can provide consistent and predictable production and transfer of carbonated beverages or other gasified liquids with controlled amounts of dissolved gasses. This implementation can be useful in the home brew and small micro brew industries but not necessarily limited to these industries. This implementation can be used in, but not restricted to, the production of beer or other carbonated liquid where the process of fermentation of dissolved sugars in solution are converted to primarily carbon dioxide gas and ethyl alcohol. The generated carbon dioxide gas, if not allowed to escape to the atmosphere, is the primary gas used for the carbonation of the liquid. This process is commonly referred as “natural conditioning,” and may be employed in commercial breweries as well as non-commercial breweries such as in home-brew processes. Other methods of carbonation include direct injection of carbon dioxide gas or a liquid or solid that releases carbon dioxide gas as part of a chemical reaction. Direct injection carbonation may be more costly and more time consuming than using a controlled natural conditioning process and the end product is different in character when carbon dioxide injection is used as opposed to natural conditioning.

In accordance with another implementation, where the transfer of liquid containing a controlled amount of dissolved gas under pressure is transferred to another vessel. The transfer process uses gas pressure to move the liquid and a pressure relief valve on the receiving vessel which is set to maintain consistent pressure on the liquid throughout the transfer process. It is important to maintain consistent pressure during the transfer process because a loss of pressure while transferring carbonated liquids, especially beer, may result in a loss of gas from solution and excessive foaming. The amount of foam depends upon the amount of pressure drop and the rate at which the carbonated liquid is transferred, possibly generating a volcano-like reaction with foam oozing from the vent or spitting from the relief valve. This is not only detrimental to the beer or gasified liquid but can produce a sizeable mess before the transfer is complete.

This is one of the major reasons why the transfer of carbonated liquids, especially beer, is avoided in the home brew and smaller micro brew operations. And for this reason, most home brewers may not get into producing naturally conditioned beverages in bulk, which would most likely require a vessel-to-vessel transfer at some point because the process of natural conditioning also produces organic byproducts that form sediment in the bottom of the vessel. A quality finished product should be free of sediment which can cloud the finished product and in some cases produce off flavors. Manual intervention to control this process is possible, difficult, and subject to errors and problems as stated above. Using calibrated relief valves with fixed springs of various tensions or relief valves with adjustable spring tension and pressurized gas to move the liquid are the solution to the problem of pressure loss or over pressurization during the transfer of liquids containing dissolved gasses.

### BRIEF DESCRIPTION OF THE DRAWINGS

The accompanying drawings, which are incorporated in and constitute a part of this specification, illustrate an embodiment of the invention and, together with the description, explain the invention.

FIG. 1A illustrates a set of springs, consistent with the principles of the invention;

FIG. 1B illustrates a relief valve assembly, exploded view;

FIG. 1C illustrates an assembled relief valve;

FIGS. 2A and 2B illustrate an adjustable relief valve assembly;

FIG. 3A illustrates an access cover for a typical pressure vessel, top view, showing a relief valve;

FIG. 3B illustrates an access cover for a typical pressure vessel, side view, showing a relief valve;

FIG. 4 illustrates a pressure vessel, top view, showing access cover with relief valve; and

FIG. 5 illustrates a double pressure vessel setup for the transfer of liquids containing dissolved gas.

### DETAILED DESCRIPTION

In the drawings, springs of various tensions are used. In certain cases springs can be used with existing relief valve housings, such as a relief valve found on pressure vessels used for beverages. When used with existing housings, the existing spring is removed and replaced with a new spring of known tension and pressure relief. This replacement will change the purpose of the pressure relief valve from that of a safety release valve which prevents the vessel from exploding to that of a calibrated pressure release valve which controls the amount of absorbed gas in solution. Since the release pressures of the calibrated valve are substantially lower than that of the original safety valve, safety does not become an issue with the retrofit.

By selecting a spring of a known tension or adjusting spring tension against the relief valve, the operator can control the amount of dissolved gasses in a liquid that is contained in a vessel and carbonated with a process from within as in natural conditioning, but not necessarily limited to natural conditioning. Amounts of dissolved gasses will vary based on gas type, liquid, temperature and pressure. Gas solubility tables exist and need to be referenced to determine the correct pressure for a given gas/liquid environment at a given temperature. In the case of carbonated liquids, the gas is carbon dioxide and the liquid is water or a water-based solution. The source of carbon dioxide for the natural conditioning of carbonated beverages is quite often a particular strain of yeast dissolved in a sugary, water-based solution which is allowed to ferment. During the fermentation process the yeast will produce primarily carbon dioxide and ethyl alcohol. The carbon dioxide, if contained and kept under pressure, will be absorbed into the liquid. Absorbed amounts of carbon dioxide will vary based on temperature of the solution, the maximum pressure allowed and the amount of time these conditions are held within the containment vessel.

In another implementation, removable relief valve parts, on a commonly available pressure vessel, are replaced with an assembly that enables the operator to adjust the valve's relief pressure within a given range without changing springs. Instead, the operator will simply make a tension adjustment on the supplied spring to vary the relief pressure within the spring's variable capacity. Expanding the relief pressure range may exceed the capacity of one spring and require replacing the original spring with another spring with a higher or lower range of tension.

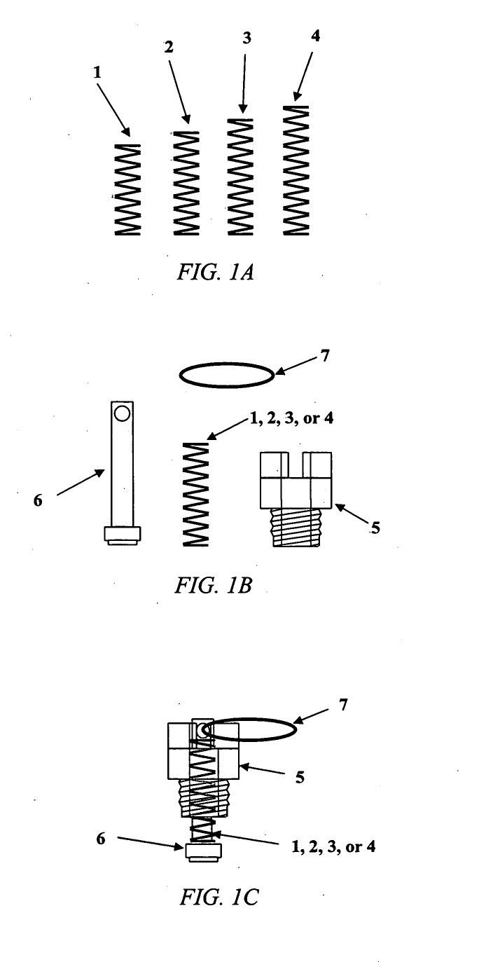

This invention will now be described more fully hereinafter with reference to the accompanying drawings, in which preferred embodiments of the invention are shown. This invention may however be embodied in many different forms and should not be construed as limited to the embodiments set forth herein. These embodiments are provided so that this disclosure will convey the scope of the invention to those who are skilled in the art. Referring to FIG. 1A, By way of example, the spring with the shortest length 1 is capable of producing a force equivalent to a pressure relief of approximately 8 p.s.i.; the next longer spring 2 is capable of producing a force equivalent to a pressure relief of approximately 12 p.s.i.; the next longer spring 3, 16 p.s.i.; and the longest 4, 20 p.s.i.

Referring to FIG. 1A, a set of springs according to one embodiment of the invention is depicted and used in conjunction with a set of relief valve parts FIG. 1B on a low volume pressure vessel used in the beverage industry. Referring to FIG. 1C, The selected spring is placed over the shaft of valve seal 6 followed by the base 5 which will compress the spring and be held in place by a retaining device such as a split ring 7 which is inserted through the hole in the end of the shaft on valve seal 6.

Relative to each other, the shorter spring having less tension against the valve seal 6 will maintain a lower pressure inside the vessel. For example, the shortest spring 1 may have an equivalent pressure regulation of 8 p.s.i., the next longer 2 12 p.s.i., and so on, giving the operator a choice of maximum internal vessel pressure thereby controlling the amount of dissolved gas in the liquid within the vessel. An operator trained in the art of natural conditioning of carbonated beverages would have the tools and knowledge necessary to select the correct relief pressure for the beverage product in production.

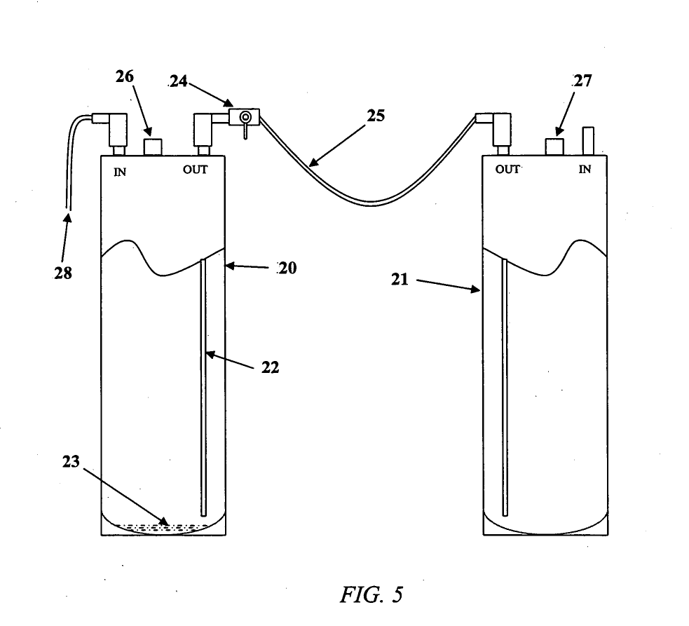

In another implementation, using FIG. 1C as an example, would be for, but not limited to, the controlled pressure transfer of carbonated liquid. As illustrated in FIG. 5, this process would use 2 pressure vessels of similar size and design, one being the sending vessel 20 and the other, a receiving vessel 21. The sending vessel 20 would contain the naturally conditioned liquid including the associated precipitates and sediments 23 which are settled out on the bottom of the vessel. The discharge tube 22 inside the sending vessel may be sized to draw liquid from above the sediment 23, thereby leaving the undesirable sediments 23 behind and transferring only clear liquid.

The sending vessel's relief valve 26 may be established at a minimum of 2-5 p.s.i. greater than the receiving vessel's relief valve 27, which is set to the same internal pressure as the sending vessel 20 before the transfer begins. The receiving vessel 21 would normally be purged of air or at least receive a quantity of gas (e.g. carbon dioxide) that would blanket the liquid during transfer and be pressurized to the same level as the sending vessel 20. Pressurized gas is connected to the inlet 28 of the sending vessel 20. Gas pressure may be regulated and held to 2-3 p.s.i. above the sending vessel's internal pressure. Opening the stop valve 24 will start the transfer process. The receiving vessel 21 will vent excess gas pressure automatically through the relief valve as it fills, thereby maintaining pressure and the original level of soluble gas in the liquid.

For example, if the established sending vessel 20 pressure is 12 p.s.i., the relief valve 26 of the sending vessel 20 would need to be retrofitted with a 16 p.s.i. spring 3 while the receiving vessel's relief valve 27 is retrofitted with a 12 p.s.i. spring 2. Vessels would be connected “out” to “out” (discharge tube to discharge tube) with a stop valve 24, clear tubing 25 and associated connectors which are purged with gas (e.g. carbon dioxide) to eliminate air/oxygen contamination of the product. Note: When there are no “in” or “out” connections made on the vessel, pressure is maintained by spring-loaded stop valves in the vessel's connectors. The “in” connection on the sending vessel would be connected to a regulated gas source 28 with pressure set to approximately 13-14 p.s.i.

As the stop valve 24 is opened, the liquid starts flowing and begins to over pressurize the receiving vessel 21 but its relief valve 27 starts to open and keeps the pressure at the set 12 p.s.i. level. At the end of the transfer, the gas that has displaced the liquid in the sending vessel 20 will begin to flow up the discharge tube 22 of the sending vessel becoming visible as large bubbles in the interconnect tubing 25. At this time the operator would stop the transfer process by closing the stop valve 24. The receiving vessel 21 now contains the finished liquid/gas product free from sediment 23 and foam and a gas blanket of carbon dioxide pressurized to 12 p.s.i. The product is now ready for use (e.g. consumption).

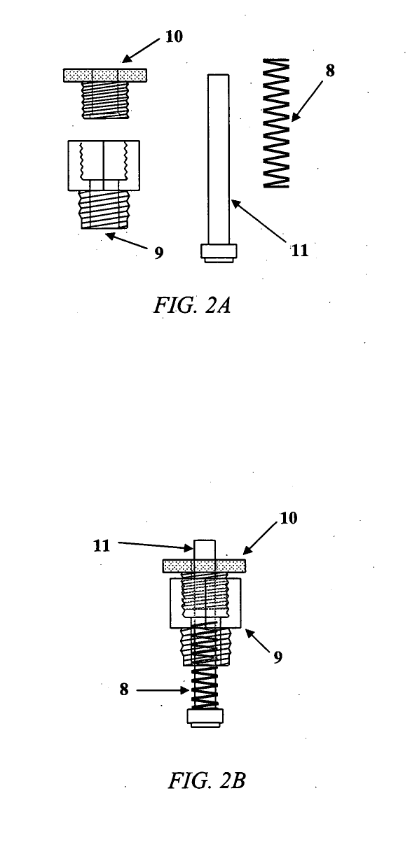

Referring to FIG. 2A and FIG. 2B, procedures for producing naturally conditioned beverages and transferring that product to another pressure vessel are identical to those depicted above except that the original relief valve assemblies 26 and 27 in the pressure vessels 20 and 21 are replaced by the adjustable relief valve assembly shown in FIG. 2B. Correct pressure settings can be established for these adjustable relief valves by using an empty pressure vessel retrofitted with the adjustable relief valve FIG. 2B set to maximum pressure regulation, pressure gauge, and filling the vessel with a test gas or air to the desired pressure. Start reducing the spring tension on the relief valve and continue the reduction until gas just begins to escape from the valve and then slightly increase spring tension until the gas stops escaping and maintains the desired pressure.

FIG. 1A illustrates a set of pressure control springs used to replace the standard safety relief spring in many of the pressure vessels used in small volume production of carbonated beverages by home brewers and micro breweries. These vessels may be retrofitted from soft drink syrup containers commonly used in soda fountains and are still used today. But over the years these discarded, used kegs have been adopted by the home brew industry for production, storage and dispensing of beer or other carbonated beverages. In general, the springs in the set can be identical in design except for their length. When using the same wire diameter for spring production, the longer springs provide a higher level of pressure regulation when compressed inside identical pressure relief assemblies. This is due to the additional spring tension generated from the additional compression.

FIG. 1B illustrates a set of the relief valve's removable parts, typically found on many of the smaller pressure vessels. Substituting one of the springs in FIG. 1A for the original spring will allow the operator to select a much lower relief pressure, thereby automatically controlling the maximum vessel pressure and the amount of dissolved gas in solution inside the pressure vessel.

FIG. 1C illustrates an assembled pressure relief valve. This assembly may be used to replace an existing safety relief valve assembly typically found on 5 gallon soda kegs, where operating pressures may reach up to 120 p.s.i.

FIGS. 2A and 2B illustrate an adjustable pressure relief valve which can replace the original pressure vessel relief valve's removable parts. This assembly shows one of several designs that would typically be used on many of the pressure vessels in use today. It allows the operator to select a wide range of pressures simply by tightening (screwing in) the tension adjustment to increase pressure or loosening (unscrewing) the tension adjustment to reduce pressure.

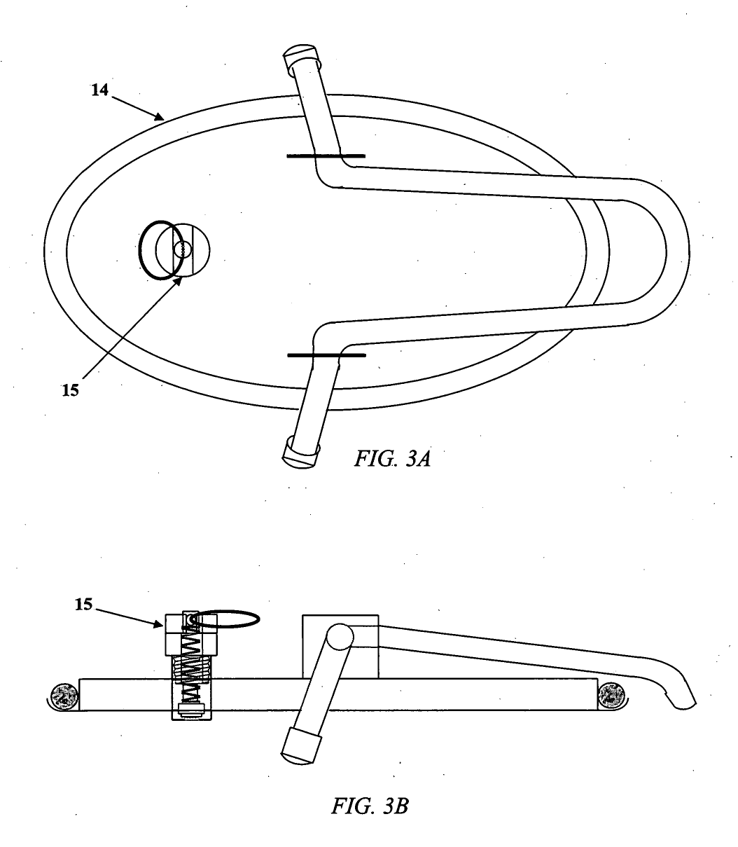

FIG. 3A illustrates a pressure vessel access cover 14, top view, showing an installed relief valve 15.

FIG. 3B illustrates a pressure vessel access cover 14, side view, showing an installed relief valve 15.

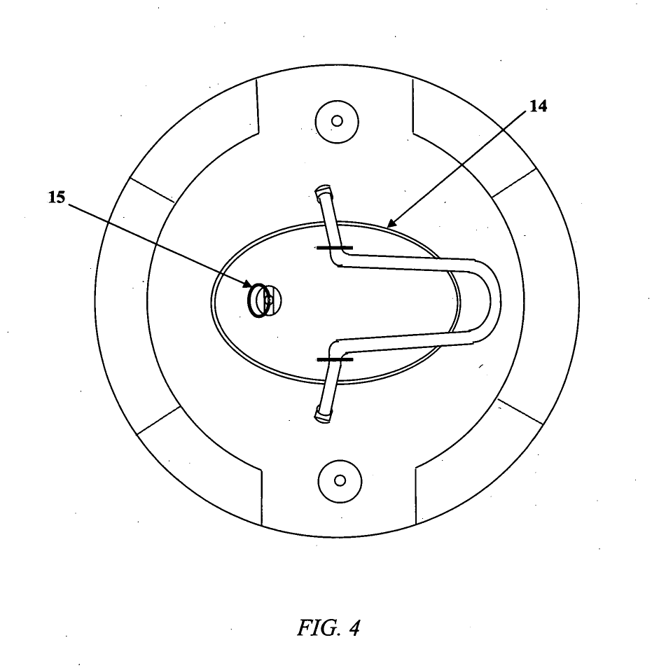

FIG. 4 illustrates a pressure vessel's top view with access cover 14 and relief valve 15 installed on a vessel, such as a vessel used to store a pressurized beverage.

FIG. 5 illustrates two pressure vessels set up for a tank-to-tank transfer of carbonated liquid.

### CONCLUSION

Implementations consistent with the invention make possible for the operator to easily and automatically control, during production, or maintain during transfer, the amount of dissolved gas in a liquid through controlled pressure regulation.

The scope of the invention is defined by the claims and their equivalents.

What is claimed:

1. A method for regulating pressure for a vessel containing a fermenting liquid for the purpose of regulating the amount of dissolved gas in the liquid, comprising:
identifying a desired pressure for the vessel while the liquid ferments, where the desired pressure maintains a predetermined amount of gas in solution;
installing a spring into a relief valve on the vessel, where the spring can be tensioned to maintain the desired pressure in the vessel when the spring is maintained in the relief valve;
venting excess pressure in the vessel automatically by having the spring change from a predetermined compressed length to a shorter compressed length when the pressure in the vessel exceeds the desired pressure, where the shorter compressed length allows excess pressure to escape the vessel until the desired pressure is reached; and
sealing the vessel when the desired pressure is reached by having the spring return to the predetermined compressed length.

2. A pressure relief assembly to maintain a desired pressure in a vessel containing a fermenting liquid where the desired pressure aids in the regulation of maximum dissolved gas, carbon dioxide or carbonation in the liquid during fermentation or maintaining a constant pressure during liquid transfer to another vessel, the assembly comprising:
a housing having an inner portion exposed to the desired pressure when the carbonated liquid is in the vessel and for supporting a lower end of a pressure relief device, the housing also having an outer portion for retaining an upper portion of a spring-based pressure relief device in a determined position; and
a pressure relief device having a lower portion supportable by the inner portion of the housing and an upper portion supported against the outer portion of the housing, the spring arranged to maintain the desired pressure by compressing when the pressure in the vessel exceeds the desired pressure, where the compressing allows excess pressure to exit the vessel until the desired pressure is achieved.

3. A system for transferring a carbonated or gasified liquid from a source vessel to a receiving vessel while maintaining a consistent pressure on the liquid during the transfer process to the receiving vessel, the system comprising:
a source vessel having a pressure relief assembly that includes a spring having a known tension, for example, that regulates a pressure 2-5 psi above a pressure in the receiving vessel;
a receiving vessel having a pressure relief assembly that includes a spring having a known tension that maintains a desired pressure in the receiving vessel during liquid transfer;
a regulated pressurized gas source to move the gasified liquid from the source vessel to the receiving vessel; and
a transfer hose connected between an outlet connection of the source vessel to an outlet connection of the receiving vessel to transport the gasified liquid from the source vessel to the receiving vessel.
